(19) United States
(12) Patent Application Publication
(10) Pub. No.: US 2025/0262285 A1
(43) Pub. Date: Aug. 21, 2025

(54) Topical Formulations for Reducing Bruising and/or Pain

(71) Applicant: Andrew Sternlicht, Boston, MA (US)

(72) Inventor: Andrew Sternlicht, Boston, MA (US)

(21) Appl. No.: 18/909,495

(22) Filed: Oct. 8, 2024

Related U.S. Application Data
(63) Continuation of application No. 18/589,611, filed on Feb. 28, 2024, now abandoned.
Application No. 18/589,611 is a continuation of application No. 18/225,786, filed on Jul. 25, 2023, now abandoned.
Application No. 18/225,786 is a continuation of application No. 18/075,854, filed on Dec. 6, 2022, now abandoned.
Application No. 18/075,854 is a continuation of application No. 16/227,581, filed on Dec. 20, 2018, now abandoned.
(60) Provisional application No. 62/608,514, filed on Dec. 20, 2017.

(51) Int. Cl. A61K 38/48; A61K 9/00; A61K 31/352; A61K 36/15; A61K 36/28; A61P 17/02

(52) U.S. Cl. CPC A61K 38/4873; A61K 9/0014; A61K 9/0053; A61K 31/352; A61K 36/15; A61K 36/28; A61P 17/02; C12Y 304/22032

(57) ABSTRACT

Provided herein are methods and compositions useful for treating bruising, trauma, and/or pain, or other symptoms associated therewith. These compositions and methods are useful in reducing the size, color, or swelling of a bruise, which may be associated with surgery and/or another form of trauma.

### CROSS REFERENCE TO RELATED APPLICATION

The present application is a continuation of U.S. application Ser. No. 18/589,611, filed on Feb. 28, 2024, which is a continuation of U.S. application Ser. No. 18/225,786, filed on Jul. 25, 2023, which is a continuation of U.S. application Ser. No. 18/075,854, filed on Dec. 6, 2022, which is a continuation of U.S. application Ser. No. 16/227,581, filed on Dec. 20, 2018, which claims priority under 35 U.S.C. § 119(e) to U.S. Provisional Application No. 62/608,514, filed Dec. 20, 2017, the entire contents of which are incorporated herein by reference.

### BACKGROUND OF THE INVENTION

Bruising after surgery or trauma is a common response which results from capillary rupture or blood collection in and around a postoperative site or site of trauma. Bruises typically last 7 to 10 days, but can persist for several weeks. Bruises are commonly associated with swelling as blood collecting in tissues incites an inflammatory response, and in surgery, particularly in cosmetic surgery, this can increase tension on a fresh postoperative incision, which may lead to poor healing or a more visible postoperative scar. Bruises are associated with increased post operative pain and delay the ability of a patient to return to normal activities and interactions. Bruising is also commonly associated with the subcutaneous injection of fillers or collagen or sometimes even following botulinum toxin injection.

Anecdotal treatments for bruising include the use of either oral dietary supplements or topical application of naturally derived products, such as bromelain, arnica, or quercertin. While multiple studies have examined the efficacy of these agents for the treatment of various skin ailments, such as, for example, the use of high concentrations (e.g., 35%) of bromelain to promote debridement of dead tissue after severe burns, randomized trials using varying concentrations of arnica have been inconclusive in determining its efficacy. In addition, the U.S. Food and Drug Administration (FDA) has issued a variety of warnings that these natural products (e.g., quercetin, apigenin, and bromelain) are not regulated and can be potentially harmful due to the lack of clinical trials and data to substantiate their benefits.

### SUMMARY OF THE INVENTION

Bruising after surgery or trauma can increase pain, cause poor wound healing, potentially increase the likelihood of infection, and delay the ability of a patient to return to normal interactions, as well as potentially having a psychosocial impact on the patient. Reduction of post-operative or post-traumatic bruising and its sequelae is an integral aspect of promoting healing and advancing the patient recovery process. To accomplish this goal, the efficacy of arnica, bromelain, quercetin, and/or apigenin on treating bruising, pain, and/or trauma has been evaluated for the first time in a systematic, dose-dependent manner. Thus, the present disclosure provides compositions and methods useful for treating bruising, pain, and/or trauma.

In one aspect, the present disclosure provides compositions comprising, consisting essentially of, or consisting of one or more agents selected from arnica, bromelain, quercetin, and apigenin. In certain embodiments, the composition comprises, two or more, three or more, or all four agents selected from arnica, bromelain, quercetin, and apigenin. In certain embodiments, the composition further comprises an agent that increases transdermal absorption. In certain embodiments, the composition further comprises DMSO. In certain embodiments, the composition further comprises one or more anesthetic agents selected from prilocaine, lidocaine, benzocaine, tetracaine, and proxymetacaine. In certain embodiments, the composition further comprises lidocaine. In certain embodiments, the composition further comprises prilocaine. In certain embodiments, the composition further comprises lidocaine and prilocaine. In certain embodiments, the composition further comprises a topically acceptable adjuvant or emollient.

In another aspect, the present disclosure provides compositions comprising, consisting essentially of, or consisting of one or more agents selected from arnica, bromelain, quercetin, and apigenin for use in treating bruising, trauma, and/or pain. In a further aspect, the present invention provides use of the compositions comprising, consisting essentially of, or consisting of one or more agents selected from arnica, bromelain, quercetin, and apigenin to treat bruising, trauma, and/or pain in a subject in need thereof.

In a further aspect, the compositions described herein may be formulated for topical and/or transdermal administration. In some embodiments, the composition is formulated as a cream, ointment, lotion, patch, or aerosol for direct administration to the site of bruising, trauma, and/or pain. In some embodiments, a composition described herein is formulated for oral administration. In some embodiments, the composition is formulated as a pill, capsule, or tablet.

In another aspect, the present disclosure provides methods for treating bruising and/or trauma in a tissue sample, wherein the site of bruising and/or trauma is contacted directly with a composition described herein. In some embodiments, the tissue sample is ex vivo. In some embodiments, the tissue sample is in vivo.

In a further aspect, the present disclosure provides methods for treating bruising and/or trauma in a subject, wherein the site of bruising and/or trauma is directly contacted with a composition described herein. In some embodiments, a composition described herein is orally administered to a subject in need thereof. In some embodiments, the subject has undergone surgery. In some embodiments, a composition described herein is administered pre-operatively, post-operatively, or a combination of both.

In yet a further aspect, provided herein are compositions and methods for reducing the size, swelling, or color of a bruise and/or other symptoms associated with bruising and/or trauma. In some embodiments, inflammation at the site of bruising, trauma, and/or pain is reduced. In some embodiments, the rate of healing of a bruise and/or other symptom associated with bruising and/or trauma is increased.

Also provided herein are pharmaceutical compositions and kits comprising, consisting essentially of, or consisting of one or more agents selected from arnica, bromelain, quercetin, apigenin, and optionally an agent that increases the transdermal absorption of the composition, an anesthetic agent, and/or a pharmaceutically acceptable adjuvant, excipient, or emollient.

All patents, patent applications, books, articles, documents, databases, websites, publications, references, etc., mentioned herein are incorporated by reference in their entirety. In case of a conflict between the specification and any of the incorporated references, the specification (including any amendments thereof) shall control. Applicants reserve the right to amend the specification based, e.g., on any of the incorporated material and/or to correct obvious errors. None of the content of the incorporated material shall limit the invention. Standard art-accepted meanings of terms are used herein unless indicated otherwise. Standard abbreviations for various terms are used herein.

The details of certain embodiments of the invention are set forth in the Detailed Description of Certain Embodiments, as described below. Other features, objects, and advantages of the invention will be apparent from the Definitions, Examples, Figures, and Claims.

### Definitions

Descriptions and certain information relating to various terms used in the present disclosure are collected herein for convenience.

The term “agent” is used herein to refer to any substance, compound (e.g., molecule), supramolecular complex, material, or combination or mixture thereof. The term “compound” and “agent” are used interchangeably. A compound may be any agent that can be represented by a chemical formula, chemical structure, or sequence. Example of agents, include, for example, small molecules, polypeptides, nucleic acids (e.g., RNAi agents, antisense oligonucleotide, aptamers), lipids, polysaccharides, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments, the agent is a naturally derived product (e.g., a naturally occurring enzyme). In some embodiments, an agent may be present in a natural extract. In some embodiments, the natural extract is derived from any part of a plant. In some embodiments, the plant is a pineapple (Ananas cososus). In some embodiments, the plant is a member of the sunflower family (e.g., Arnica montana, or a related Arnica species). In some embodiments an agent is cell-permeable, e.g., within the range of typical agents that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates [e.g., with water (i.e., hydrates) or common solvents] and/or may have different crystalline forms (e.g., polymorphs) or different tautomeric forms. Embodiments exhibiting such alternative protonation states, configurations, solvates, and forms are encompassed by the present disclosure where applicable.

The terms “composition” and “formulation” may be used interchangeably, unless explicitly stated. A composition may comprise one or more active ingredients. In certain embodiments, the active ingredient is arnica. In certain embodiments, the active ingredient is bromelain.

A “percentage concentration” denotes a concentration of a compound based on the total volume or weight of a solution. The total volume or weight of a solution is the sum of the volume or weight of the solute plus the total volume or weight of the solvent. Percentage concentrations may be denoted as volume per volume (v/v), weight per volume (w/v), or weight per weight (w/w). In general, % v/v is used for solutions in which both the solute and solvent are expressed in terms of volumes (e.g., milliliters). In general, % w/v is used for solutions in which a solute is expressed in terms of weight (e.g., milligrams) is dissolved in a solvent expressed in terms of volume (e.g., milliliters). In general, % w/w is used for solution in which both the solute and solvent are expressed in terms of their weight (e.g., milligrams). It should be noted that where the total concentration is given as a weight/weight (w/w) concentration, the equivalent volume/weight (v/w) and/or volume/volume (v/v) concentration is also contemplated.

The terms “assess,” “determine,” “evaluate,” and “assay” are used interchangeably herein to refer to any form of detection or measurement, and include determining whether a substance, signal, disease, condition, etc., is present or not. The result of an assessment may be expressed in qualitative and/or quantitative terms. Assessing may be relative or absolute. “Assessing the presence of” includes determining the amount of something that is present or determining whether it is present or absent.

The term “sample” may be used to generally refer to an amount or portion of something. The sample may be a “biological sample”. A sample may be a smaller quantity taken from a larger amount or entity; however, a complete specimen may also be referred to as a sample where appropriate. A sample is often intended to be similar to and representative of a larger amount of the entity of which it is a sample. In some embodiments a sample is a quantity of a substance that is or has been or is to be provided for assessment (e.g., testing, analysis, measurement) or use. A sample may be any biological specimen. In some embodiments a sample comprises a body fluid such as blood, cerebrospinal fluid, (CSF), sputum, lymph, mucus, saliva, a glandular secretion, or urine. In some embodiments a sample comprises cells, tissue, or cellular material (e.g., material derived from cells, such as a cell lysate or fraction thereof). A sample may be obtained from (i.e., originates from, was initially removed from) a subject. Methods of obtaining biological samples from subjects are known in the art and include, e.g., tissue biopsy, such as excisional biopsy, incisional biopsy, core biopsy; fine needle aspiration biopsy; surgical excision; brushings; lavage; or collecting body fluids that may contain cells, such as blood, sputum, lymph, mucus, saliva, or urine. A sample is often intended to be similar to and representative of a larger amount of the entity of which it is a sample. In some embodiments a sample is obtained from skin or blood. In some embodiments a sample contains at least some intact cells. In some embodiments a sample retains at least some of the microarchitecture of a tissue from which it was removed. A sample may be subjected to one or more processing steps, e.g., after having been obtained from a subject, and/or may be split into one or more portions. The term sample encompasses processed samples, portions of samples, etc., and such samples are, where applicable, considered to have been obtained from the subject from whom the initial sample was removed. A sample may be procured directly from a subject, or indirectly, e.g., by receiving the sample from one or more persons who procured the sample directly from the subject, e.g., by performing a biopsy, surgery, or other procedure on the subject. In certain embodiments, the sample is an abdominoplasty tissue sample obtained intraoperatively (i.e., carried out during the course of surgery). In certain embodiments, the sample is a blood sample obtained intraoperatively.

The term “target tissue” refers to any biological tissue of a subject (including a group of cells, a body part, or an organ) or a part thereof, including blood and/or lymph vessels, which is the object to which a compound, particle, and/or composition of the invention is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is a tissue comprising one or more bruises or sites of trauma.

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. The phrases “effective amount” and “therapeutically effective amount” are used interchangeably. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that cells or tissues may be contacted with an “effective amount”, or an “effective amount” may be administered to a subject in a single dose, or through use of multiple doses, in various embodiments. In certain embodiments, an effective amount is an amount of a composition that reduces the size, color, and swelling of a bruise compared to an untreated bruise. In certain embodiments, the site of bruising or trauma is contacted directly with a composition described herein. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for reducing bruising. In certain embodiments, a therapeutically effective amount is an amount sufficient for reducing the size, color, or swelling of a bruise. In certain embodiments, a therapeutically effective amount is an amount sufficient for reducing pain. In certain embodiments, a therapeutically effective amount is an amount sufficient for treating bruising, pain, and/or trauma.

A “subject” may be any vertebrate organism in various embodiments. A subject may be individual to whom an agent is administered, e.g., for experimental, diagnostic, and/or therapeutic purposes or from whom a sample is obtained or on whom a procedure is performed. A “subject” to which administration is contemplated refers to a human (i.e., male or female of any age group, e.g., pediatric subject (e.g., infant, child, or adolescent) or adult subject (e.g., young adult, middle-aged adult, or senior adult)). In certain embodiments, a subject is a human that has undergone surgery. In certain embodiment, a subject is a human that has undergone trauma. In certain embodiments, the subject has one or more sites of bruising as a result of surgery and/or trauma.

The term “administer,” “administering,” or “administration” refers to implanting, absorbing, ingesting, injecting, inhaling, applying, or otherwise introducing a compound described herein, or a composition thereof, in or on a subject.

“Treat,” “treating” and similar terms as used herein in the context of treating a subject refer to providing medical and/or surgical management of a subject. Treatment may include, but is not limited to, administering a composition (e.g., a pharmaceutical composition) to a subject. Treatment is typically undertaken in an effort to alter the course of a condition or disease (which term is used to indicate any disease, disorder, syndrome or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the condition or disease or one or more symptoms or manifestations associated with the condition or disease. A therapeutic agent may be administered to a subject who has a condition or disease or is at increased risk of developing a condition or disease relative to a member of the general population. In certain embodiments, the condition to be treated is bruising resulting from surgery and/or trauma. In certain embodiments, the condition to be treated is a painful condition or pain associated with surgery or trauma. The agent or composition may be administered pre-operatively, e.g., to reduce the likelihood of bruising (i.e., prophylactic treatment). The agent or composition may be administered post-operatively or post-traumatically, i.e., after surgery or trauma has occurred. In some embodiments, a first composition is administered pre-operatively and a second composition is administered post-operatively, where the first and the second compositions are any of the compositions described herein.

### BRIEF DESCRIPTION OF THE DRAWINGS

The accompanying drawings, which constitute a part of this specification, illustrate exemplary embodiments of the invention and together with the description, serve to explain certain principles of the invention. The embodiments disclosed in the drawings are exemplary and do not limit the scope of this disclosure.

FIG. 1 shows the control discoloration in an abdominoplasty specimen after injection of 25 μL of patient blood intradermally.

### DETAILED DESCRIPTION OF CERTAIN EMBODIMENTS

Compositions for Treating Bruising and/or Pain

Some aspects of this invention are based at least in part on the fact that agents including arnica, bromelain, quercetin, and apigenin provide beneficial therapeutic properties that can be employed for the treatment of bruising, pain, and or other symptoms associated with trauma. Specifically, the compositions provided herein comprise agents that, either alone or in combination, can provide anti-inflammatory, antifibrinolytic, and/or antioxidant properties.

Some aspects of this invention provide compositions comprising, consisting essentially of, or consisting of arnica. Throughout the present disclosure, the term “arnica” may refer to any agent (e.g., enzyme, compound, small molecule, fatty acid, oil, etc.) derived from the Arnica genus of flowering plants. The term “arnica” may also encompass whole-plant (e.g., Arnica montana) extracts. In some embodiments, the source of arnica is Arnica montana. Arnica montana contains essential oils, fatty acids, thymol, pseudoguanianolide sesquiterpene lactones, such as helenalin and fatty ester derivatives thereof, and flavanone glycosides. Any of these Arnica montana constituents may provide one or more useful therapeutic properties when arnica is present in a composition described herein. For example, without wishing to be bound by any particular theory, arnica may confer anti-inflammatory properties when present in a composition. The arnica used in various embodiments of the present disclosure may be obtained from any source. Commercially available forms of arnica derived from Arnica montana include powder forms and oil extracts. Arnica may be obtained from the any part of the Arnica montana plant using a liquid-solid extraction or solvent extraction method, which are known in the art. In some embodiments, a composition herein comprises arnica provided in powdered form. In some embodiments, the arnica powder is pharmaceutical, medicine, and/or food grade. In some embodiments, the arnica powder comprises at least 80%, at least 85%, at least 90%, at least 95%, at least 98%, at least 99%, at least 99.5%, or at least 100% arnica (e.g., active ingredient) by weight. In some embodiments, the arnica powder comprises at least 95% arnica (e.g., active ingredient) by weight). In some embodiments, the arnica powder comprises at least 98% arnica (e.g., active ingredient) by weight). In some embodiments, the arnica powder comprises at least 99% arnica (e.g., active ingredient) by weight). In some embodiments, the arnica powder comprises at least 99.5% arnica (e.g., active ingredient) by weight. In general, the arnica powder can be dissolved in, dispersed in, or mixed with a pharmaceutically acceptable adjuvant, carrier, or excipient to provide a formulation for topical administration.

In some embodiments, the composition comprises, consists essentially of, or consists of a total arnica concentration of about 0% (w/w) to about 25% (w/w). The total arnica concentration is based on the addition of arnica powder to a pharmaceutically acceptable adjuvant, excipient, or emollient, such that the total concentration of arnica (e.g., active ingredient) in the final composition is between about 0-25% (w/w). In some embodiments, the arnica powder comprises less than 100% arnica, and thus more powder will need to be added to the pharmaceutically acceptable adjuvant, excipient, or emollient to provide a composition with the appropriate (w/w) concentration of arnica. Thus, in some embodiments, the total arnica concentration does not include any excipients or ingredients other than arnica that may be present in the arnica powder used in the composition. In some embodiments, the total arnica concentration in the composition is about 0-25% (w/w), including any decimal amount therebetween. In some embodiments, the total arnica concentration in the composition is about 0-5% (w/w), including any decimal amount therebetween. In some embodiments, the total arnica concentration in the composition is about 5-10% (w/w), including any decimal amount therebetween. In some embodiments, the total arnica concentration in the composition is about 10-15% (w/w), including any decimal amount therebetween. In some embodiments, the total arnica concentration in the composition is about 15-20% (w/w), including any decimal amount therebetween. In some embodiments, the total arnica concentration in the composition is about 20-25% (w/w), including any decimal amount therebetween. In some embodiments, the total arnica concentration in the composition is about 0% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19%, (w/w), 20% (w/w), 21% (w/w), 22% (w/w), 23% (w/w), 24% (w/w), or 25% (w/w). In some embodiments, the total arnica concentration in the composition is about 0% (w/w), 5% (w/w), 10% (w/w), 15% (w/w), 20% (w/w), or 25% (w/w). In some embodiments, the total arnica concentration in the composition is about 5% (w/w), 10% (w/w), or 20% (w/w). In some embodiments, the total arnica concentration in the composition is about 1% (w/w). In some embodiments, the total arnica concentration in the composition is about 5% (w/w). In some embodiments, the total arnica concentration in the composition is about 10% (w/w). In some embodiments, the total arnica concentration in the composition is about 20% (w/w).

Some aspects of this invention provide compositions comprising, consisting essentially of, or consisting of bromelain. Throughout the present disclosure, the term “bromelain” refers to an enzyme extract derived from pineapples (Ananas comosus). In some embodiments, the bromelain is derived from the stems of pineapples (i.e., the source). The term “bromelain” encompasses the stem bromelain enzyme (EC 3.4.22.32), the fruit bromelain enzyme (EC 3.4.22.33), or a combination of the stem and fruit bromelain enzymes, and optionally other compounds present in the extract. In addition, the term “bromelain” may also refer to the crude, unseparated mixture of proteolytic enzymes extracted from pineapple stems. Bromelain may provide one or more useful therapeutic properties when present in a composition described herein. For example, without wishing to be bound by any particular theory, bromelain may confer antifibrinolytic and/or proteolytic properties when present in a composition. The bromelain used in various embodiments of the present disclosure may be obtained from any source. Commercially available forms of bromelain derived from pineapple include powder forms and oil extracts. Bromelain may be obtained from the any part of the pineapple (e.g., the stem) using a liquid-solid extraction or solvent extraction method, which are known in the art. In some embodiments, a composition herein comprises bromelain provided in powdered form. In some embodiments, the bromelain powder is cosmetic, pharmaceutical, medicine, and/or food grade. In some embodiments, the bromelain powder comprises at least 80%, at least 85%, at least 90%, at least 95%, at least 98%, at least 99%, at least 99.5%, or at least 100% bromelain (e.g., active ingredient) by weight. In some embodiments, the bromelain powder comprises at least 95% bromelain (e.g., active ingredient) by weight). In some embodiments, the bromelain powder comprises at least 98% bromelain (e.g., active ingredient) by weight). In some embodiments, the bromelain powder comprises at least 99% bromelain (e.g., active ingredient) by weight). In some embodiments, the bromelain powder comprises at least 99.5% bromelain (e.g., active ingredient) by weight). In general, the bromelain powder can be dissolved in, dispersed in, or mixed with a pharmaceutically acceptable adjuvant, carrier, or excipient to provide a formulation for topical administration.

In some embodiments, the composition comprises, consists essentially of, or consists of a total bromelain concentration of about 0% (w/w) to about 25% (w/w). The total bromelain concentration is based on the addition of bromelain powder to a pharmaceutically acceptable adjuvant, excipient, or emollient, such that the total concentration of bromelain (e.g., active ingredient) in the final composition is between about 0-25% (w/w). In some embodiments, the bromelain powder comprises less than 100% bromelain, and thus more powder will need to be added to the pharmaceutically acceptable adjuvant, excipient, or emollient to provide a composition with the appropriate (w/w) concentration of bromelain. Thus, in some embodiments, the total bromelain concentration does not include any excipients or ingredients other than bromelain that may be present in the bromelain powder used in the composition. In some embodiments, the total concentration of bromelain does not exceed 35% (w/w). In some embodiments, the total bromelain concentration in the composition is about 0-25% (w/w), including any decimal amount therebetween. In some embodiments, the total bromelain concentration in the composition is about 0-10% (w/w), including any decimal amount therebetween. In some embodiments, the total bromelain concentration in the composition is about 10-20% (w/w), including any decimal amount therebetween. In some embodiments, the total bromelain concentration in the composition is about 0-5% (w/w), including any decimal amount therebetween. In some embodiments, the total bromelain concentration in the composition is about 5-10% (w/w), including any decimal amount therebetween. In some embodiments, the total bromelain concentration in the composition is about 0-1% (w/w), including any decimal amount therebetween. In some embodiments, the total bromelain concentration in the composition is about 0% (w/w), 0.5% (w/w), 1% (w/w), 1.5% (w/w), 2% (w/w), 2.5% (w/w), 3% (w/w), 3.5% (w/w), 4% (w/w), 4.5% (w/w), 5% (w/w), 5.5% (w/w), 6% (w/w), 6.5% (w/w), 7% (w/w), 7.5% (w/w), 8% (w/w), 8.5% (w/w), 9% (w/w), 9.5%, (w/w), 10% (w/w), 15% (w/w), 20% (w/w), or 25% (w/w). In some embodiments, the total bromelain concentration in the composition is about 0% (w/w), 0.5% (w/w), 1% (w/w), 1.5% (w/w), 2% (w/w), 5% (w/w), or 10% (w/w). In some embodiments, the total bromelain concentration in the composition is about 0.5% (w/w), 1% (w/w), or 5% (w/w). In some embodiments, the total bromelain concentration in the composition is about 0.5% (w/w). In some embodiments, the total bromelain concentration in the composition is about 1% (w/w). In some embodiments, the total bromelain concentration in the composition is about 5% (w/w).

Some aspects of this invention provide compositions comprising, consisting essentially of, or consisting of quercetin. Quercetin is a plant polyphenol found in many fruits, vegetables, leaves, grains, and other plant parts. The term “quercetin” also encompasses the terms 2-(3,4-dihydroxyphenyl)-3,5,7-trihydroxy-4H-chromen-4-one, 5,7,3′,4′-flavon-3-ol, sophoretin, meletin, quercetine, xanthaurine, quercetol, quercitin, quertine, and flavin meletin. Quercetin may provide one or more useful therapeutic properties when present in a composition described herein. For example, without wishing to be bound by any particular theory, quercetin may confer anti-inflammatory and/or antioxidant properties when present in a composition. The quercetin used in various embodiments of the present disclosure may be obtained from any source. Quercetin is commonly found in many different foods, for example, capers, onion, radish, sweet potato, broccoli, and kidney beans, among others. Quercetin may also be found in the flowers of plants, such as, for example, Styphnolobium (sophora) japonica. Commercially available forms of quercetin derived from the above mentioned plant sources, or any other plant that contains quercetin, include powder forms and oil extracts. Quercetin may be obtained from the any part of the plant (e.g., the flowers) using a liquid-solid extraction or solvent extraction method, which are known in the art. In some embodiments, a composition herein comprises quercetin provided in powdered form. In some embodiments, the quercetin powder is pharmaceutical, medicine, and/or food grade. In some embodiments, the quercetin powder comprises at least 80%, at least 85%, at least 90%, at least 95%, at least 98%, at least 99%, at least 99.5%, or at least 100% quercetin (e.g., active ingredient) by weight. In some embodiments, the quercetin powder comprises at least 95% quercetin (e.g., active ingredient) by weight). In some embodiments, the quercetin powder comprises at least 98% quercetin (e.g., active ingredient) by weight). In some embodiments, the quercetin powder comprises at least 99% quercetin (e.g., active ingredient) by weight). In some embodiments, the quercetin powder comprises at least 99.5% quercetin (e.g., active ingredient) by weight). In general, the quercetin powder can be dissolved in, dispersed in, or mixed with a pharmaceutically acceptable adjuvant, carrier, or excipient to provide a formulation for topical administration.

In some embodiments, the composition comprises, consists essentially of, or consists of a total quercetin concentration of about 0% (w/w) to about 10% (w/w). The total quercetin concentration is based on the addition of quercetin powder to a pharmaceutically acceptable adjuvant, excipient, or emollient, such that the total concentration of quercetin (e.g., active ingredient) in the final composition is between about 0-25% (w/w). In some embodiments, the quercetin powder comprises less than 100% quercetin, and thus more powder will need to be added to the pharmaceutically acceptable adjuvant, excipient, or emollient to provide a composition with the appropriate (w/w) concentration of quercetin. Thus, in some embodiments, the total quercetin concentration does not include any excipients or ingredients other than quercetin that may be present in the quercetin powder used in the composition. In some embodiments, the total concentration of quercetin does not exceed 10% (w/w). In some embodiments, the total concentration of quercetin does not exceed 3% (w/w). In some embodiments, the total quercetin concentration in the composition is about 0-10% (w/w), including any decimal amount therebetween. In some embodiments, the total quercetin concentration in the composition is about 0.1-10% (w/w), including any decimal amount therebetween. In some embodiments, the total quercetin concentration in the composition is about 0.1-5% (w/w), including any decimal amount therebetween. In some embodiments, the total quercetin concentration in the composition is about 0.1-3% (w/w), including any decimal amount therebetween. In some embodiments, the total quercetin concentration in the composition is about 0.1-1% (w/w), including any decimal amount therebetween. In some embodiments, the total quercetin concentration in the composition is about 0.1-0.5% (w/w), including any decimal amount therebetween. In some embodiments, the total quercetin concentration in the composition is about 0% (w/w), 0.1% (w/w), 0.2 (w/w), 0.3% (w/w), 0.4% (w/w), 0.5% (w/w), 1% (w/w), 1.5% (w/w), 2% (w/w), 2.5% (w/w), 3% (w/w), 3.5% (w/w), 4% (w/w), 4.5% (w/w), 5% (w/w), 5.5% (w/w), 6% (w/w), 6.5% (w/w), 7% (w/w), 7.5% (w/w), 8% (w/w), 8.5% (w/w), 9% (w/w), 9.5%, (w/w), or 10% (w/w). In some embodiments, the total quercetin concentration in the composition is about 0% (w/w), 0.1% (w/w), 0.5% (w/w), 1% (w/w), 1.5% (w/w), 2% (w/w), 2.5% (w/w), or 3% (w/w). In some embodiments, the total quercetin concentration is about 2% (w/w), 2.5% (w/w), or 3% (w/w). In some embodiments, the total quercetin concentration in the composition is about 0.1% (w/w), 0.5% (w/w), or 1% (w/w). In some embodiments, the total quercetin concentration in the composition is about 0.1% (w/w). In some embodiments, the total quercetin concentration in the composition is about 0.5% (w/w). In some embodiments, the total quercetin concentration in the composition is about 1% (w/w).

Some aspects of this invention provide compositions comprising, consisting essentially of, or consisting of apigenin. Apigenin is a flavone found in many plants and parts of plants (e.g., flowers). Sources of apigenin include, for example, parsley, celery, celeriac, and chamomile. The term “apigenin” also encompasses the terms 5,7-dihydroxy-2-(4-hydroxyphenyl)-4H-1-benzopyran-4-one, apigenine, chamomile, apigenol, spigenin, versulin, 4′,5,7-trihydroxyflavone, and C.I. natural yellow 1. Apigenin may provide one or more useful therapeutic properties when present in a composition described herein. For example, without wishing to be bound by any particular theory, apigenin may confer anti-inflammatory and/or antioxidant properties when present in a composition. The apigenin used in various embodiments of the present disclosure may be obtained from any source. Commercially available forms of apigenin derived from the above mentioned plant sources, or any other plant source which contains apigenin, include powder forms and oil extracts. Apigenin may be obtained from the any part of the plant using a liquid-solid extraction or solvent extraction method, which are known in the art. In some embodiments, a composition herein comprises apigenin provided in powdered form. In some embodiments, the apigenin powder is pharmaceutical, medicine, and/or food grade. In some embodiments, the apigenin powder comprises at least 80%, at least 85%, at least 90%, at least 95%, at least 98%, at least 99%, at least 99.5%, or at least 100% apigenin (e.g., active ingredient) by weight. In some embodiments, the apigenin powder comprises at least 95% apigenin (e.g., active ingredient) by weight). In some embodiments, the apigenin powder comprises at least 98% apigenin (e.g., active ingredient) by weight). In some embodiments, the apigenin powder comprises at least 99% apigenin (e.g., active ingredient) by weight). In some embodiments, the apigenin powder comprises at least 99.5% apigenin (e.g., active ingredient) by weight). In general, the apigenin powder can be dissolved in, dispersed in, or mixed with a pharmaceutically acceptable adjuvant, carrier, or excipient to provide a formulation for topical administration.

In some embodiments, the composition comprises, consists essentially of, or consists of a total apigenin concentration of about 0% (w/w) to about 25% (w/w). The total apigenin concentration is based on the addition of apigenin powder to a pharmaceutically acceptable adjuvant, excipient, or emollient, such that the total concentration of apigenin (e.g., active ingredient) in the final composition is between about 0-25% (w/w). In some embodiments, the apigenin powder comprises less than 100% apigenin, and thus more powder will need to be added to the pharmaceutically acceptable adjuvant, excipient, or emollient to provide a composition with the appropriate (w/w) concentration of apigenin. Thus, in some embodiments, the total apigenin concentration does not include any excipients or ingredients other than apigenin that may be present in the apigenin powder used in the composition. In some embodiments, the total concentration of apigenin does not exceed 10% (w/w). In some embodiments, the total concentration of apigenin does not exceed 3% (w/w). In some embodiments, the total apigenin concentration in the composition is about 0-10% (w/w), including any decimal amount therebetween. In some embodiments, the total apigenin concentration in the composition is about 0.1-10% (w/w), including any decimal amount therebetween. In some embodiments, the total apigenin concentration in the composition is about 0.1-5% (w/w), including any decimal amount therebetween. In some embodiments, the total apigenin concentration in the composition is about 0.1-3% (w/w), including any decimal amount therebetween. In some embodiments, the total apigenin concentration in the composition is about 0.1-1% (w/w), including any decimal amount therebetween. In some embodiments, the total apigenin concentration in the composition is about 0.1-0.5% (w/w), including any decimal amount therebetween. In some embodiments, the total apigenin concentration in the composition is about 0% (w/w), 0.1% (w/w), 0.2 (w/w), 0.3% (w/w), 0.4% (w/w), 0.5% (w/w), 1% (w/w), 1.5% (w/w), 2% (w/w), 2.5% (w/w), 3% (w/w), 3.5% (w/w), 4% (w/w), 4.5% (w/w), 5% (w/w), 5.5% (w/w), 6% (w/w), 6.5% (w/w), 7% (w/w), 7.5% (w/w), 8% (w/w), 8.5% (w/w), 9% (w/w), 9.5%, (w/w), or 10% (w/w). In some embodiments, the total apigenin concentration in the composition is about 0% (w/w), 0.1% (w/w), 0.5% (w/w), 1% (w/w), 1.5% (w/w), 2% (w/w), 2.5% (w/w), or 3% (w/w). In some embodiments, the total apigenin concentration is about 2% (w/w), 2.5% (w/w), or 3% (w/w). In some embodiments, the total apigenin concentration in the composition is about 0.1% (w/w), 0.5% (w/w), or 1% (w/w). In some embodiments, the total apigenin concentration in the composition is about 0.1% (w/w). In some embodiments, the total apigenin concentration in the composition is about 0.5% (w/w). In some embodiments, the total apigenin concentration in the composition is about 1% (w/w).

In some embodiments, the compositions described herein may further comprise an agent that increases the transdermal absorption of the composition. Agents that increase the transdermal absorption or permeation through a biological membrane (e.g., the skin) are discussed in Aungst BJ (2012) Absorption Enhancers: Applications and Advances, AAPS J, 14(1): pp. 10-18, which is herein incorporated by reference. Some examples of agents that may increase the transdermal absorption of a composition are cyclopentadecalactone (pentadecalactone), dimethyl sulfoxide (DMSO), salcaprozate sodium (SNAC), and other pharmaceutically acceptable excipients, adjuvants, and emollients. In some embodiments, the agent that increases transdermal absorption is DMSO.

In some embodiments, the compositions described herein may further comprise one or more anesthetic agents. “Anesthetic agents” are agents that reduce or prevent pain. For example, local anesthetic agents cause a limited, reversible loss of sensation in a limited region (i.e., where locally applied). A local anesthetic agent suitable for use herein may be, for example, procaine, amethocaine, lidocaine, prilocaine, bupivacaine, levobupivacaine, ropivacaine, mepivacaine, dibucaine, benzocaine, tetracaine, or proxymetacaine. The anesthetic agents used herein may be added to a pharmaceutically acceptable adjuvant, excipient, or emollient in solid form (e.g., from a powder), liquid form (e.g., from a cream). In some embodiments, the composition further comprises one or more anesthetic agents. In some embodiments, the composition further comprises one or more anesthetic agents selected from lidocaine, prilocaine, benzocaine, tetracaine, and proxymetacaine. In some embodiments, the composition further comprises lidocaine. In some embodiments, the composition further comprises prilocaine. In some embodiments, the composition further comprises lidocaine and priolocaine. In some embodiments, the composition further comprises an anesthetic agent at a total concentration of between about 0% (w/w) and about 20% (w/w). The total concentration of anesthetic can be the concentration of a single anesthetic agent, or the combined concentration of two anesthetic agents, three anesthetic agents, four anesthetic agents, or five anesthetic agents. In some embodiments, the total concentration of anesthetic is the concentration of a single anesthetic. In some embodiments, the total concentration of anesthetic is the combined concentration of two anesthetic agents. For example, in some embodiments, the total concentration of lidocaine in the composition is 2.5% (w/w) and the total concentration of prilocaine in the composition is 2.5% (w/w). In some embodiments, the total anesthetic concentration in the composition is about 0-20% (w/w), including any decimal amount therebetween. In some embodiments, the total anesthetic concentration in the composition is about 0-10% (w/w), including any decimal amount therebetween. In some embodiments, the total anesthetic concentration in the composition is about 10-20% (w/w), including any decimal amount therebetween. In some embodiments, the total anesthetic concentration in the composition is about 0-5% (w/w), including any decimal amount therebetween. In some embodiments, the total anesthetic concentration in the composition is about 0% (w/w), 0.5% (w/w), 1% (w/w), 1.5% (w/w), 2% (w/w), 2.5% (w/w), 3% (w/w), 3.5% (w/w), 4% (w/w), 4.5% (w/w), 5% (w/w), 5.5% (w/w), 6% (w/w), 6.5% (w/w), 7% (w/w), 7.5% (w/w), 8% (w/w), 8.5% (w/w), 9% (w/w), 9.5%, (w/w), 10% (w/w), 15% (w/w), or 20% (w/w). In some embodiments, the total anesthetic concentration in the composition is about 1% (w/w), 2% (w/w), or 5% (w/w). In some embodiments, the total anesthetic concentration in the composition is about 1% (w/w). In some embodiments, the total anesthetic concentration in the composition is about 2% (w/w). In some embodiments, the total anesthetic concentration in the composition is about 5% (w/w).

In some embodiments, the composition comprises, consists essentially of, or consists of a total lidocaine concentration of about 0% (w/w) to about 10% (w/w). In some embodiments, the total lidocaine concentration in the composition is about 0-10% (w/w), including any decimal amount therebetween. In some embodiments, the total lidocaine concentration in the composition is about 0-5% (w/w), including any decimal amount therebetween. In some embodiments, the total lidocaine concentration in the composition is about 0-2.5% (w/w), including any decimal amount therebetween. In some embodiments, the total lidocaine concentration in the composition is about 0-1% (w/w), including any decimal amount therebetween. In some embodiments, the total lidocaine concentration in the composition is about 0% (w/w), 0.5% (w/w), 1.0% (w/w), 1.5% (w/w), 2.0% (w/w), 2.5% (w/w), 3% (w/w), 3.5% (w/w), 4.0% (w/w), 4.5% (w/w), or 5% (w/w). In some embodiments, the total lidocaine concentration in the composition is about 0.5% (w/w). In some embodiments, the total lidocaine concentration in the composition is about 1.0% (w/w). In some embodiments, the total lidocaine concentration in the composition is about 1.5% (w/w). In some embodiments, the total lidocaine concentration in the composition is about 2.5% (w/w).

In some embodiments, the composition comprises, consists essentially of, or consists of a total prilocaine concentration of about 0% (w/w) to about 10% (w/w). In some embodiments, the total prilocaine concentration in the composition is about 0-10% (w/w), including any decimal amount therebetween. In some embodiments, the total prilocaine concentration in the composition is about 0-5% (w/w), including any decimal amount therebetween. In some embodiments, the total prilocaine concentration in the composition is about 0-2.5% (w/w), including any decimal amount therebetween. In some embodiments, the total prilocaine concentration in the composition is about 0-1% (w/w), including any decimal amount therebetween. In some embodiments, the total prilocaine concentration in the composition is about 0% (w/w), 0.5% (w/w), 1.0% (w/w), 1.5% (w/w), 2.0% (w/w), 2.5% (w/w), 3% (w/w), 3.5% (w/w), 4.0% (w/w), 4.5% (w/w), or 5% (w/w). In some embodiments, the total prilocaine concentration in the composition is about 0.5% (w/w). In some embodiments, the total prilocaine concentration in the composition is about 1.0% (w/w). In some embodiments, the total prilocaine concentration in the composition is about 1.5% (w/w). In some embodiments, the total prilocaine concentration in the composition is about 2.5% (w/w).

In one aspect, the present disclosure provides a composition comprising, consisting essentially of, or consisting of one or more agents selected from arnica, bromelain, quercetin, and apigenin. In some embodiments, the composition comprises, consists essentially of, or consists of arnica. In some embodiments, the composition comprises, consists essentially of, or consists of bromelain. In some embodiments, the composition comprises, consists essentially of, or consists of quercetin. In some embodiments, the composition comprises, consists essentially of, or consists of apigenin.

In a further aspect, the present disclosure provides a composition further comprising an agent that increases the transdermal absorption of the composition. In some embodiments, the composition further comprises DMSO. In some embodiments, the composition comprises, consists essentially of, or consists of arnica and DMSO. In some embodiments, the composition comprises, consists essentially of, or consists of bromelain and DMSO. In some embodiments, the composition comprises, consists essentially of, or consists of quercetin and DMSO. In some embodiments, the composition comprises, consists essentially of, or consists of apigenin and DMSO.

In yet a further aspect, the composition further comprises one or more anesthetic agents selected from prilocaine, lidocaine, benzocaine, tetracaine, and proxymetacaine. In some embodiments, the composition comprises, consists essentially of, or consists of arnica and prilocaine. In some embodiments, the composition comprises, consists essentially of, or consists of bromelain and prilocaine. In some embodiments, the composition comprises, consists essentially of, or consists of quercetin and prilocaine. In some embodiments, the composition comprises, consists essentially of, or consists of apigenin and prilocaine. In some embodiments, the composition comprises, consists essentially of, or consists of arnica and lidocaine. In some embodiments, the composition comprises, consists essentially of, or consists of bromelain and lidocaine. In some embodiments, the composition comprises, consists essentially of, or consists of quercetin and lidocaine. In some embodiments, the composition comprises, consists essentially of, or consists of apigenin and lidocaine. In some embodiments, the composition comprises, consists essentially of, or consists of arnica and benzocaine. In some embodiments, the composition comprises, consists essentially of, or consists of bromelain and benzocaine. In some embodiments, the composition comprises, consists essentially of, or consists of quercetin and benzocaine. In some embodiments, the composition comprises, consists essentially of, or consists of apigenin and benzocaine. In some embodiments, the composition comprises, consists essentially of, or consists of arnica and tetracaine. In some embodiments, the composition comprises, consists essentially of, or consists of bromelain and tetracaine. In some embodiments, the composition comprises, consists essentially of, or consists of quercetin and tetracaine. In some embodiments, the composition comprises, consists essentially of, or consists of apigenin and tetracaine. In some embodiments, the composition comprises, consists essentially of, or consists of arnica and proxymetacaine. In some embodiments, the composition comprises, consists essentially of, or consists of bromelain and proxymetacaine. In some embodiments, the composition comprises, consists essentially of, or consists of quercetin and proxymetacaine. In some embodiments, the composition comprises, consists essentially of, or consists of apigenin and proxymetacaine. In some embodiments, the composition further comprises an agent that increases the transdermal absorption of the composition. In some embodiments, the composition further comprises DMSO. In some embodiments, the composition comprises, consists essentially of, or consists of arnica, prilocaine, and DMSO. In some embodiments, the composition comprises, consists essentially of, or consists of bromelain, prilocaine, and DMSO. In some embodiments, the composition comprises, consists essentially of, or consists of quercetin, prilocaine, and DMSO. In some embodiments, the composition comprises, consists essentially of, or consists of apigenin, prilocaine, and DMSO. In some embodiments, the composition comprises, consists essentially of, or consists of arnica, lidocaine, and DMSO. In some embodiments, the composition comprises, consists essentially of, or consists of bromelain, lidocaine, and DMSO. In some embodiments, the composition comprises, consists essentially of, or consists of quercetin, lidocaine, and DMSO. In some embodiments, the composition comprises, consists essentially of, or consists of apigenin, lidocaine, and DMSO.

In some embodiments, the composition further comprises two or more anesthetic agents selected from prilocaine, lidocaine, benzocaine, tetracaine, and proxymetacaine. In some embodiments, the composition comprises, consists essentially of, or consists of arnica, prilocaine, and lidocaine. In some embodiments, the composition comprises, consists essentially of, or consists of bromelain, prilocaine, and lidocaine. In some embodiments, the composition comprises, consists essentially of, or consists of quercetin, prilocaine, and lidocaine. In some embodiments, the composition comprises, consists essentially of, or consists of apigenin, prilocaine, and lidocaine. In some embodiments, the composition comprises, consists essentially of, or consists of arnica, prilocaine, lidocaine, and DMSO. In some embodiments, the composition comprises, consists essentially of, or consists of bromelain, prilocaine, lidocaine, and DMSO. In some embodiments, the composition comprises, consists essentially of, or consists of quercetin, prilocaine, lidocaine, and DMSO. In some embodiments, the composition comprises, consists essentially of, or consists of apigenin, prilocaine, lidocaine, and DMSO.

In another aspect, the present disclosure provides a composition comprising, consisting essentially of, or consisting of two or more agents selected from arnica, bromelain, quercetin, and apigenin. In some embodiments, the composition comprises, consists essentially of, or consists of arnica and bromelain. In some embodiments, the composition comprises, consists essentially of, or consists of arnica and quercetin. In some embodiments, the composition comprises, consists essentially of, or consists of arnica and apigenin. In some embodiments, the composition comprises, consists essentially of, or consists of bromelain and quercetin. In some embodiments, the composition comprises, consists essentially of, or consists of bromelain and apigenin. In some embodiments, the composition comprises, consists essentially of, or consists of quercetin and apigenin. In some embodiments, the composition further comprises an agent that increases the transdermal absorption of the composition. In some embodiments, the composition further comprises DMSO. In some embodiments, the composition comprises, consists essentially of, or consists of arnica, bromelain, and DMSO. In some embodiments, the composition comprises, consists essentially of, or consists of arnica, quercetin, and DMSO. In some embodiments, the composition comprises, consists essentially of, or consists of arnica, apigenin, and DMSO. In some embodiments, the composition comprises, consists essentially of, or consists of bromelain, quercetin, and DMSO. In some embodiments, the composition comprises, consists essentially of, or consists of bromelain, apigenin, and DMSO. In some embodiments, the composition comprises, consists essentially of, or consists of quercetin, apigenin, and DMSO.

In a further aspect, the present disclosure provides compositions further comprising one or more anesthetic agents selected from prilocaine, lidocaine, benzocaine, tetracaine, and proxymetacaine. In some embodiments, the composition comprises, consists essentially of, or consists of arnica, bromelain, and prilocaine. In some embodiments, the composition comprises, consists essentially of, or consists of arnica, quercetin, and prilocaine. In some embodiments, the composition comprises, consists essentially of, or consists of arnica, apigenin, and prilocaine. In some embodiments, the composition comprises, consists essentially of, or consists of bromelain, quercetin, and prilocaine. In some embodiments, the composition comprises, consists essentially of, or consists of bromelain, apigenin, and prilocaine. In some embodiments, the composition comprises, consists essentially of, or consists of quercetin, apigenin, and prilocaine. In some embodiments, the composition comprises, consists essentially of, or consists of arnica, bromelain, and lidocaine. In some embodiments, the composition comprises, consists essentially of, or consists of arnica, quercetin, and lidocaine. In some embodiments, the composition comprises, consists essentially of, or consists of arnica, apigenin, and lidocaine. In some embodiments, the composition comprises, consists essentially of, or consists of bromelain, quercetin, and lidocaine. In some embodiments, the composition comprises, consists essentially of, or consists of bromelain, apigenin, and lidocaine. In some embodiments, the composition comprises, consists essentially of, or consists of quercetin, apigenin, and lidocaine. For the sake of brevity, all possible combinations of one or more anesthetic agents in combination with two or more agents selected from arnica, bromelain, quercetin, and apigenin are not being parsed out. In some embodiments, the composition further comprises an agent that increases the transdermal absorption of the composition. In some embodiments, the composition further comprises DMSO. In some embodiments, the composition comprises, consists essentially of, or consists of arnica, bromelain, and prilocaine. In some embodiments, the composition comprises, consists essentially of, or consists of arnica, quercetin, prilocaine, and DMSO. In some embodiments, the composition comprises, consists essentially of, or consists of arnica, apigenin, prilocaine, and DMSO. In some embodiments, the composition comprises, consists essentially of, or consists of bromelain, quercetin, prilocaine, and DMSO. In some embodiments, the composition comprises, consists essentially of, or consists of bromelain, apigenin, prilocaine, and DMSO. In some embodiments, the composition comprises, consists essentially of, or consists of quercetin, apigenin, prilocaine, and DMSO. In some embodiments, the composition comprises, consists essentially of, or consists of arnica, bromelain, lidocaine, and DMSO. In some embodiments, the composition comprises, consists essentially of, or consists of arnica, quercetin, lidocaine, and DMSO. In some embodiments, the composition comprises, consists essentially of, or consists of arnica, apigenin, lidocaine, and DMSO. In some embodiments, the composition comprises, consists essentially of, or consists of bromelain, quercetin, lidocaine, and DMSO. In some embodiments, the composition comprises, consists essentially of, or consists of bromelain, apigenin, lidocaine, and DMSO. In some embodiments, the composition comprises, consists essentially of, or consists of quercetin, apigenin, lidocaine, and DMSO.

In some embodiments, the composition further comprises two or more anesthetic agents selected from prilocaine, lidocaine, benzocaine, tetracaine, and proxymetacaine. In some embodiments, the composition comprises, consists essentially of, or consists of arnica, bromelain, prilocaine, and lidocaine. In some embodiments, the composition comprises, consists essentially of, or consists of arnica, quercetin, prilocaine, and lidocaine. In some embodiments, the composition comprises, consists essentially of, or consists of arnica, apigenin, prilocaine, and lidocaine. In some embodiments, the composition comprises, consists essentially of, or consists of bromelain, quercetin, prilocaine, and lidocaine. In some embodiments, the composition comprises, consists essentially of, or consists of bromelain, apigenin, prilocaine, and lidocaine. In some embodiments, the composition comprises, consists essentially of, or consists of quercetin, apigenin, prilocaine, and lidocaine. For the sake of brevity, all possible combinations of two or more anesthetic agents in combination with two or more agents selected from arnica, bromelain, quercetin, and apigenin are not being parsed out. In some embodiments, the composition further comprises an agent that increases the transdermal absorption of the composition. In some embodiments, the composition further comprises DMSO. In some embodiments, the composition comprises, consists essentially of, or consists of arnica, bromelain, prilocaine, lidocaine, and DMSO. In some embodiments, the composition comprises, consists essentially of, or consists of arnica, quercetin, prilocaine, lidocaine, and DMSO. In some embodiments, the composition comprises, consists essentially of, or consists of arnica, apigenin, prilocaine, lidocaine, and DMSO. In some embodiments, the composition comprises, consists essentially of, or consists of bromelain, quercetin, prilocaine, lidocaine, and DMSO. In some embodiments, the composition comprises, consists essentially of, or consists of bromelain, apigenin, prilocaine, lidocaine, and DMSO. In some embodiments, the composition comprises, consists essentially of, or consists of quercetin, apigenin, prilocaine, lidocaine, and DMSO.

In another aspect, the present disclosure provides a composition comprising, consisting essentially of, or consisting of three or more agents selected from arnica, bromelain, quercetin, and apigenin. In some embodiments, the composition comprises, consists essentially of, or consists of arnica, bromelain, and quercetin. In some embodiments, the composition comprises, consists essentially of, or consists of arnica, bromelain, and apigenin. In some embodiments, the composition comprises, consists essentially of, or consists of arnica, quercetin, and apigenin. In some embodiments, the composition comprises, consists essentially of, or consists of bromelain, quercetin, and apigenin. In some embodiments, the composition further comprises an agent that increases the transdermal absorption of the composition. In some embodiments, the composition further comprises DMSO. In some embodiments, the composition comprises, consists essentially of, or consists of arnica, bromelain, quercetin, and DMSO. In some embodiments, the composition comprises, consists essentially of, or consists of arnica, bromelain, apigenin, and DMSO. In some embodiments, the composition comprises, consists essentially of, or consists of arnica, quercetin, apigenin, and DMSO. In some embodiments, the composition comprises, consists essentially of, or consists of bromelain, quercetin, apigenin, and DMSO.

In a further aspect, the present disclosure provides compositions further comprising one or more anesthetic agents selected from prilocaine, lidocaine, benzocaine, tetracaine, and proxymetacaine. In some embodiments, the composition comprises, consists essentially of, or consists of arnica, bromelain, quercetin, and prilocaine. In some embodiments, the composition comprises, consists essentially of, or consists of arnica, bromelain, apigenin, and prilocaine. In some embodiments, the composition comprises, consists essentially of, or consists of arnica, quercetin, apigenin, and prilocaine. In some embodiments, the composition comprises, consists essentially of, or consists of bromelain, quercetin, apigenin, and prilocaine. In some embodiments, the composition comprises, consists essentially of, or consists of arnica, bromelain, quercetin, and lidocaine. In some embodiments, the composition comprises, consists essentially of, or consists of arnica, bromelain, apigenin, and lidocaine. In some embodiments, the composition comprises, consists essentially of, or consists of arnica, quercetin, apigenin, and lidocaine. In some embodiments, the composition comprises, consists essentially of, or consists of bromelain, quercetin, apigenin, and lidocaine. For the sake of brevity, all possible combinations of one or more anesthetic agents in combination with three or more agents selected from arnica, bromelain, quercetin, and apigenin are not being parsed out. In some embodiments, the composition further comprises an agent that increases the transdermal absorption of the composition. In some embodiments, the composition further comprises DMSO. In some embodiments, the composition comprises, consists essentially of, or consists of arnica, bromelain, quercetin, prilocaine, and DMSO. In some embodiments, the composition comprises, consists essentially of, or consists of arnica, bromelain, apigenin, prilocaine, and DMSO. In some embodiments, the composition comprises, consists essentially of, or consists of arnica, quercetin, apigenin, prilocaine, and DMSO. In some embodiments, the composition comprises, consists essentially of, or consists of bromelain, quercetin, apigenin, prilocaine, and DMSO. In some embodiments, the composition comprises, consists essentially of, or consists of arnica, bromelain, quercetin, lidocaine, and DMSO. In some embodiments, the composition comprises, consists essentially of, or consists of arnica, bromelain, apigenin, lidocaine, and DMSO. In some embodiments, the composition comprises, consists essentially of, or consists of arnica, quercetin, apigenin, lidocaine, and DMSO. In some embodiments, the composition comprises, consists essentially of, or consists of bromelain, quercetin, apigenin, lidocaine, and DMSO.

In some embodiments, the composition further comprises two or more anesthetic agents selected from prilocaine, lidocaine, benzocaine, tetracaine, and proxymetacaine. In some embodiments, the two agents are prilocaine and lidocaine. In some embodiments, the composition comprises, consists essentially of, or consists of arnica, bromelain, quercetin, prilocaine, and lidocaine. In some embodiments, the composition comprises, consists essentially of, or consists of arnica, bromelain, apigenin, prilocaine, and lidocaine. In some embodiments, the composition comprises, consists essentially of, or consists of arnica, quercetin, apigenin, prilocaine, and lidocaine. In some embodiments, the composition comprises, consists essentially of, or consists of bromelain, quercetin, apigenin, prilocaine, and lidocaine. For the sake of brevity, all possible combinations of two or more anesthetic agents in combination with three or more agents selected from arnica, bromelain, quercetin, and apigenin are not being parsed out. In some embodiments, the composition further comprises an agent that increases the transdermal absorption of the composition. In some embodiments, the composition further comprises DMSO. In some embodiments, the composition comprises, consists essentially of, or consists of arnica, bromelain, quercetin, prilocaine, lidocaine, and DMSO. In some embodiments, the composition comprises, consists essentially of, or consists of arnica, bromelain, apigenin, prilocaine, lidocaine, and DMSO. In some embodiments, the composition comprises, consists essentially of, or consists of arnica, quercetin, apigenin, prilocaine, lidocaine, and DMSO. In some embodiments, the composition comprises, consists essentially of, or consists of bromelain, quercetin, apigenin, prilocaine, lidocaine, and DMSO.

In another aspect, the present disclosure provides a composition comprising, consisting essentially of, or consisting of all four agents selected from arnica, bromelain, quercetin, and apigenin. In some embodiments, the composition further comprises an agent that increases the transdermal absorption of the composition. In some embodiments, the composition further comprises DMSO. In some embodiments, the composition comprises, consists essentially of, or consists of arnica, bromelain, quercetin, apigenin, and DMSO.

In a further aspect, the present disclosure provides compositions further comprising one or more anesthetic agents selected from prilocaine, lidocaine, benzocaine, tetracaine, and proxymetacaine. In some embodiments, the composition comprises, consists essentially of, or consists of arnica, bromelain, quercetin, apigenin, and prilocaine. In some embodiments, the composition comprises, consists essentially of, or consists of arnica, bromelain, quercetin, apigenin, and lidocaine. For the sake of brevity, all possible combinations of one or more anesthetic agents in combination with all four agents selected from arnica, bromelain, quercetin, and apigenin are not being parsed out. In some embodiments, the composition further comprises an agent that increases the transdermal absorption of the composition. In some embodiments, the composition further comprises DMSO. In some embodiments, the composition comprises, consists essentially of, or consists of arnica, bromelain, quercetin, apigenin, prilocaine, and DMSO. In some embodiments, the composition comprises, consists essentially of, or consists of arnica, bromelain, quercetin, apigenin, lidocaine, and DMSO.

In some embodiments, the compositions further comprises two or more anesthetic agents selected from prilocaine, lidocaine, benzocaine, tetracaine, and proxymetacaine. In some embodiments, the composition comprises, consists essentially of, or consists of arnica, bromelain, quercetin, apigenin, prilocaine, and lidocaine. For the sake of brevity, all possible combinations of two or more anesthetic agents in combination with all four agents selected from arnica, bromelain, quercetin, and apigenin are not being parsed out. In some embodiments, the composition further comprises an agent that increases the transdermal absorption of the composition. In some embodiments, the composition further comprises DMSO. In some embodiments, the composition comprises, consists essentially of, or consists of arnica, bromelain, quercetin, apigenin, prilocaine, lidocaine, and DMSO.

In addition to the total concentration of the agents arnica, bromelain, quercetin, and/or apigenin in the composition, the ratio of active ingredients can provide a synergistic effect to increase the efficacy of the compositions for certain applications as described herein. Where a composition comprises more than one agent selected from arnica, bromelain, quercetin, and apigenin, a synergistic relationship may exist between two of the agents, three of the agent, or all four of the agents. For example, without wishing to be bound by any particular theory, compositions comprising arnica and bromelain, and optionally one or more anesthetic agents and/or an agent that increases transdermal absorption of the composition, are useful in treating bruising, trauma, and/or pain. In some embodiments, the total arnica concentration in the composition is higher than the total concentration of bromelain in the composition. In some embodiments, the concentration of arnica is between about 20 times higher to about equal to the total concentration of bromelain (i.e., between 20:1 and 1:1 arnica:bromelain). In some embodiments, the total concentration of arnica to the total concentration of bromelain is about 20:1 (i.e., 20:1 arnica:bromelain). In some embodiments, the total concentration of arnica to the total concentration of bromelain is about 15:1 (i.e., 15:1 arnica:bromelain). In some embodiments, the total concentration of arnica to the total concentration of bromelain is about 10:1 (i.e., 10:1 arnica:bromelain). In some embodiments, the total concentration of arnica to the total concentration of bromelain is about 5:1 (i.e., 5:1 arnica:bromelain). In some embodiments, the total concentration of arnica to the total concentration of bromelain is about 2:1 (i.e., 2:1 arnica:bromelain). In some embodiments, the total concentration of arnica to the total concentration of bromelain is about 1:1 (i.e., 1:1 arnica:bromelain). In some embodiments, the composition comprises, consists essentially of, or consists of a total concentration of about 5% (w/w) arnica and a total concentration of about 0.5% (w/w) bromelain. In some embodiments, the composition comprises, consists essentially of, or consists of a total concentration of about 10% (w/w) arnica and a total concentration of about 1% (w/w) bromelain. In some embodiments, the total bromelain concentration in the composition is higher than the total concentration of arnica in the composition. In some embodiments, the concentration of bromelain is between about 20 times higher to about equal to the total concentration of arnica (i.e., between 20:1 and 1:1 bromelain:arnica). In some embodiments, the total arnica concentration in the composition is higher than the total concentration of quercetin in the composition. In some embodiments, the concentration of arnica is between about 20 times higher to about equal to the total concentration of quercetin (i.e., between 20:1 and 1:1 arnica:quercetin). In some embodiments, the total quercetin concentration in the composition is higher than the total concentration of arnica in the composition. In some embodiments, the concentration of quercetin is between about 20 times higher to about equal to the total concentration of arnica (i.e., between 20:1 and 1:1 quercetin:arnica). In some embodiments, the total bromelain concentration in the composition is higher than the total concentration of quercetin in the composition. In some embodiments, the concentration of bromelain is between about 20 times higher to about equal to the total concentration of quercetin (i.e., between 20:1 and 1:1 bromelain:quercetin). In some embodiments, the total quercetin concentration in the composition is higher than the total concentration of bromelain in the composition. In some embodiments, the concentration of quercetin is between about 20 times higher to about equal to the total concentration of bromelain (i.e., between 20:1 and 1:1 quercetin:bromelain). In some embodiments, the concentration of arnica is between about 20 times higher to about equal to the total concentration of apigenin (i.e., between 20:1 and 1:1 arnica:apigenin). In some embodiments, the total apigenin concentration in the composition is higher than the total concentration of arnica in the composition. In some embodiments, the concentration of apigenin is between about 20 times higher to about equal to the total concentration of arnica (i.e., between 20:1 and 1:1 apigenin:arnica). In some embodiments, the concentration of bromelain is between about 20 times higher to about equal to the total concentration of apigenin (i.e., between 20:1 and 1:1 bromelain:apigenin). In some embodiments, the total apigenin concentration in the composition is higher than the total concentration of bromelain in the composition. In some embodiments, the concentration of apigenin is between about 20 times higher to about equal to the total concentration of bromelain (i.e., between 20:1 and 1:1 apigenin:bromelain). In some embodiments, the concentration of quercetin is between about 20 times higher to about equal to the total concentration of apigenin (i.e., between 20:1 and 1:1 quercetin:apigenin). In some embodiments, the total apigenin concentration in the composition is higher than the total concentration of quercetin in the composition. In some embodiments, the concentration of apigenin is between about 20 times higher to about equal to the total concentration of quercetin (i.e., between 20:1 and 1:1 apigenin:quercetin). Where a composition with two agents is identified, it is understood that this composition may comprise additional agents selected from arnica, bromelain, quercetin, and apigenin, anesthetic agents, agents that increase transdermal absorption, and/or a pharmaceutically acceptable adjuvant, excipient, or emollient, without effecting the synergy of the primary two agents in the composition.

In a yet a further aspect, the compositions may further comprise one or more anesthetic agents selected from prilocaine, lidocaine, benzocaine, tetracaine, and proxymetacaine. The ratio of the concentration of the anesthetic agent or the anesthetic agents to the agents selected from arnica, bromelain, quercetin, and apigenin can also provide a synergistic relationship and further improve the efficacy of the compositions described herein. For example, without wishing to be bound by any particular theory, compositions comprising a certain ratio of lidocaine and/or priolocaine to bromelain and/or arnica are useful in treating bruising, trauma, and/or pain. In some embodiments, the total concentration of arnica is higher than the total concentration of lidocaine. In some embodiments, the total concentration of arnica is between about 5 times higher to about equal to the total concentration of lidocaine (i.e., between 5:1 and 1:1 arnica:lidocaine). In some embodiments, the total concentration of arnica to the total concentration of lidocaine is about 5:1 (i.e., 5:1 arnica:lidocaine). In some embodiments, the total concentration of arnica to the total concentration of lidocaine is about 4:1 (i.e., 4:1 arnica:lidocaine). In some embodiments, the total concentration of arnica to the total concentration of lidocaine is about 3:1 (i.e., 3:1 arnica:lidocaine). In some embodiments, the total concentration of arnica to the total concentration of lidocaine is about 2:1 (i.e., 2:1 arnica:lidocaine). In some embodiments, the total concentration of arnica to the total concentration of lidocaine is about 1:1 (i.e., 1:1 arnica:lidocaine). In some embodiments, the total concentration of arnica is higher than the total concentration of prilocaine. In some embodiments, the total concentration of arnica is between about 5 times higher to about equal to the total concentration of prilocaine (i.e., between 5:1 and 1:1 arnica:prilocaine). In some embodiments, the total concentration of arnica to the total concentration of prilocaine is about 5:1 (i.e., 5:1 arnica:prilocaine). In some embodiments, the total concentration of arnica to the total concentration of prilocaine is about 4:1 (i.e., 4:1 arnica:prilocaine). In some embodiments, the total concentration of arnica to the total concentration of prilocaine is about 3:1 (i.e., 3:1 arnica:prilocaine). In some embodiments, the total concentration of arnica to the total concentration of prilocaine is about 2:1 (i.e., 2:1 arnica:prilocaine). In some embodiments, the total concentration of arnica to the total concentration of prilocaine is about 1:1 (i.e., 1:1 arnica:prilocaine). In some embodiments, the total concentration of lidocaine is higher than the total concentration of bromelain. In some embodiments, the total concentration of lidocaine is between about 5 times higher to about equal to the total concentration of bromelain (i.e., between 5:1 and 1:1 lidocaine:bromelain). In some embodiments, the total concentration of lidocaine to the total concentration of bromelain is about 5:1 (i.e., 5:1 lidocaine:bromelain). In some embodiments, the total concentration of lidocaine to the total concentration of bromelain is about 4:1 (i.e., 4:1 lidocaine:bromelain). In some embodiments, the total concentration of lidocaine to the total concentration of bromelain is about 3:1 (i.e., 3:1 lidocaine:bromelain). In some embodiments, the total concentration of lidocaine to the total concentration of bromelain is about 2:1 (i.e., 2:1 lidocaine:bromelain). In some embodiments, the total concentration of lidocaine to the total concentration of bromelain is about 1:1 (i.e., 1:1 lidocaine:bromelain). In some embodiments, the total concentration of prilocaine is higher than the total concentration of bromelain. In some embodiments, the total concentration of prilocaine is between about 5 times higher to about equal to the total concentration of bromelain (i.e., between 5:1 and 1:1 prilocaine:bromelain). In some embodiments, the total concentration of prilocaine to the total concentration of bromelain is about 5:1 (i.e., 5:1 prilocaine:bromelain). In some embodiments, the total concentration of prilocaine to the total concentration of bromelain is about 4:1 (i.e., 4:1 prilocaine:bromelain). In some embodiments, the total concentration of prilocaine to the total concentration of bromelain is about 3:1 (i.e., 3:1 prilocaine:bromelain). In some embodiments, the total concentration of prilocaine to the total concentration of bromelain is about 2.5:1 (i.e., 2.5:1 prilocaine:bromelain). In some embodiments, the total concentration of prilocaine to the total concentration of bromelain is about 2:1 (i.e., 2:1 prilocaine:bromelain). In some embodiments, the total concentration of prilocaine to the total concentration of bromelain is about 1:1 (i.e., 1:1 prilocaine:bromelain).

In yet a further aspect, a composition described herein may further comprise two or more anesthetic agents. In some embodiments, the composition further comprises two or more anesthetic agents selected from lidocaine, prilocaine, benzocaine, tetracaine, and proxymetacaine. In some embodiments, the composition further comprises lidocaine and prilocaine. In some embodiments, the total concentration of lidocaine is higher than the total concentration of prilocaine. In some embodiments, the total concentration of lidocaine is between about 5 times higher to about equal to the total concentration of prilocaine (i.e., between 5:1 and 1:1 lidocaine:prilocaine). In some embodiments, the total concentration of lidocaine to the total concentration of prilocaine is about 5:1 (i.e., 5:1 lidocaine:prilocaine). In some embodiments, the total concentration of lidocaine to the total concentration of prilocaine is about 4:1 (i.e., 4:1 lidocaine:prilocaine). In some embodiments, the total concentration of lidocaine to the total concentration of prilocaine is about 3:1 (i.e., 3:1 lidocaine:prilocaine). In some embodiments, the total concentration of lidocaine to the total concentration of prilocaine is about 2:1 (i.e., 2:1 lidocaine:prilocaine). In some embodiments, the total concentration of lidocaine is the same or about the same as the total concentration of prilocaine. In some embodiments, the total concentration of lidocaine to the total concentration of prilocaine is about 1:1 (i.e., 1:1 lidocaine:prilocaine). In some embodiments, the composition comprises or consists essentially of a total concentration of about 1% (w/w) bromelain, a total concentration of about 10% (w/w) arnica, a total concentration of about 2.5% (w/w) lidocaine, and a total concentration of about 2.5% (w/w) prilocaine. In some embodiments, the composition consists of a total concentration of about 1% (w/w) bromelain, a total concentration of about 10% (w/w) arnica, a total concentration of about 2.5% (w/w) lidocaine, and a total concentration of about 2.5% (w/w) prilocaine. In some embodiments, the total concentration of prilocaine is higher than the total concentration of lidocaine. In some embodiments, the total concentration of prilocaine is between about 5 times higher to about equal to the total concentration of lidocaine (i.e., between 5:1 and 1:1 prilocaine:lidocaine). In some embodiments, the total concentration of prilocaine to the total concentration of lidocaine is about 5:1 (i.e., 5:1 prilocaine:lidocaine). In some embodiments, the total concentration of prilocaine to the total concentration of lidocaine is about 4:1 (i.e., 4:1 prilocaine:lidocaine). In some embodiments, the total concentration of prilocaine to the total concentration of lidocaine is about 3:1 (i.e., 3:1 prilocaine:lidocaine). In some embodiments, the total concentration of prilocaine to the total concentration of lidocaine is about 2:1 (i.e., 2:1 prilocaine:lidocaine).

Methods of Treating Bruising and/or Pain

As discussed above, studies on the efficacy of arnica, bromelain, quercetin, and apigenin have provided conflicting data and results that do not clearly indicate their usefulness in treating various diseases or disorders. Studies have even suggested that homeopathic remedies, such as arnica, are not efficacious beyond a placebo effect (see Ernst E and Pittler MH (1998) Efficacy of homeopathic arnica: a systematic review of placebo-controlled clinical trials, Arch Surg, 133: 1187-1190). The compositions described herein provide the advantage of synergistic interplay between one or more ingredients and thus are useful in treating bruising, trauma, and/or pain. In some embodiments, bruising is induced by a surgical procedure. In some embodiments, bruising is induced by an injury. In some embodiments, bruising is induced by a trauma to the body. In some embodiments, pain may persist as a result of the bruising and/or trauma.

Thus, in one aspect, provided herein is are methods directed toward the treatment of bruising and other skin damage as a result of trauma. In some embodiments, the method for treating bruising and/or trauma comprises contacting the site of bruising and/or trauma of a tissue sample with an effective amount of a composition described herein. In some embodiments, the composition comprises one or more agents selected from arnica, bromelain, quercetin, and apigenin. In some embodiments, the composition further comprises an agent that increases the transdermal absorption of the composition (e.g., DMSO). In some embodiments, the composition further comprises one or more anesthetic agents selected from lidocaine, priolocaine, benzocaine, tetracaubem and proxymetacaine. In some embodiments, the tissue sample is obtained from a human subject. In some embodiments, the tissue is obtained from a patient during an abdominoplasty or other surgical procedure. In some embodiments, the tissue is contacted with a composition described herein ex vivo or in vivo.

Bruising severity may be assessed in multiple ways, including, for example, by bruise size, color, and swelling. In some embodiments, the site of bruising and/or trauma is contacted directly with a composition described herein, wherein treatment induces a reduction in bruise size. In some embodiments, the bruise size is reduced by at least 5%, 10%, 15%, 20%, 25%, 30%, 35%, 40%, 45%, 50%, 55%, 60%, 65%, 70%, 75%, 80%, 85%, 90%, or 95% upon contacting the site directly with a composition described herein. Bruise size reduction is measured in comparison to the size of the bruise before treatment with a composition described herein. In some embodiments, bruise size is reduced by at least 20% upon contacting the site directly with a composition described herein. In some embodiments, bruise size is reduced by at least 25% upon contacting the site directly with a composition described herein. In some embodiments, bruise size is reduced by at least 30% upon contacting the site directly with a composition described herein. In some embodiments, bruise size is reduced by at least 35% upon contacting the site directly with a composition described herein. In some embodiments, bruise size is reduced by at least 50% upon contacting the site directly with a composition described herein. In some embodiments, bruise size is reduced upon a single treatment (e.g., dose) with a composition described herein. In some embodiments, the site of bruising and/or trauma is contacted directly, or the single dose is administered to a subject, prior to trauma (e.g., surgery). In some embodiments, the site of trauma and/or bruising is contacted directly, or the single dose is administered to a subject, after trauma (e.g., surgery) has occurred. In some embodiments, bruise size is reduced upon multiple treatments (e.g., doses) with a composition described herein. In some embodiments, the site of bruising and/or trauma is contacted with a single dose before trauma (e.g., surgery) occurs and is again contacted with a single does after trauma (e.g., surgery) has occurred.

In some embodiments, the site of bruising and/or trauma is contacted directly with a composition described herein, wherein treatment induces a reduction in bruise color. In some embodiments, bruise color is reduced upon a single treatment (e.g., dose) with a composition described herein. In some embodiments, bruise color is reduced upon multiple treatments (e.g., doses) with a composition described herein. In some embodiments, bruise color is eliminated upon treatment with a composition described herein.

In some embodiments, the site of bruising and/or trauma is contacted directly with a composition described herein, wherein treatment induces a reduction in swelling severity. In some embodiments, swelling severity is reduced by at least 5%, 10%, 15%, 20%, 25%, 30%, 35%, 40%, 45%, 50%, 55%, 60%, 65%, 70%, 75%, 80%, 85%, 90%, or 95% upon contacting the site directly with a composition described herein. Bruise swelling reduction is measured in comparison to the swelling of the bruise before treatment with a composition described herein. In some embodiments, swelling is reduced by at least 20% upon contacting the site directly with a composition described herein. In some embodiments, swelling is reduced by at least 25% upon contacting the site directly with a composition described herein. In some embodiments, swelling is reduced by at least 30% upon contacting the site directly with a composition described herein. In some embodiments, swelling is reduced by at least 35% upon contacting the site directly with a composition described herein. In some embodiments, swelling is reduced by at least 50% upon contacting the site directly with a composition described herein. In some embodiments, swelling is reduced upon a single treatment (e.g., dose) with a composition described herein. In some embodiments, swelling is reduced upon multiple treatments (e.g., doses) with a composition described herein.

The compositions described herein comprise one or more agents that may reduce inflammation or lead to a faster rate of healing upon administration to a subject in need thereof. In some embodiments, inflammation at the site of bruising, trauma, and/or pain is reduced upon contacting the site of bruising, trauma, and/or pain with a composition described herein. In some embodiments, inflammation at the site of bruising, trauma, and/or pain is reduced upon orally administering a composition described herein to a subject. In some embodiments, the rate of healing of a bruise is increased upon contacting the site of bruising, trauma, and/or pain with a composition described herein. In some embodiments, the rate of healing of a bruise is increased upon orally administering a composition described herein to a subject.

The compositions described herein may also provide pain relief to a subject in need thereof. Pain may be a result of, for example, surgery, injury, and/or trauma to the body. In some embodiments, the site of pain is directly contacted with a composition described herein. In some embodiments, pain is reduced. In some embodiments, pain is eliminated. In some embodiments, pain is reduced upon oral administration of a composition described herein. Pain reduction can be measured in a subject by any method known in the art (see, e.g., Younger J et al. (2009) Pain Outcomes: A brief review of instruments and techniques, Curr Pain Headache Rep, 13: 39-43.), such that a physician can readily ascertain pain reduction upon treatment with a composition described herein.

In one aspect, the compositions described herein may be useful in the context of treating bruising, trauma, and/or pain in a subject who will undergo or has undergone surgery. In some embodiments, the site of bruising, trauma, and/or pain is contacted directly with a composition described herein pre-operatively. In some embodiments, the site of bruising, trauma, and/or pain is contacted directly with a composition described herein up to about one hour before surgery. In some embodiments, a composition described herein is orally administered to a subject pre-operatively. In some embodiments, a composition described herein is orally administered to a subject up to about one hour before surgery. In some embodiments, the site of bruising, trauma, and/or pain is contacted directly with a composition described herein post-operatively. In some embodiments, the site of bruising, trauma, and/or pain is contacted directly with a composition described herein immediately after surgery. In some embodiments, the site of bruising, trauma, and/or pain is contacted directly with a composition described herein about 30 minutes after surgery. In some embodiments, the site of bruising, trauma, and/or pain is contacted directly with a composition described herein up to about one hour after surgery. In some embodiments, the site of bruising, trauma, and/or pain is contacted directly with a composition described herein up to about 24 hours, 48 hours, and/or 72 hours after surgery. In some embodiments, the site of bruising, trauma, and/or pain is contacted directly with a composition described herein immediately after trauma has occurred. In some embodiments, the site of bruising, trauma, and/or pain is contacted directly with a composition described herein about 30 minutes after trauma has occurred. In some embodiments, the site of bruising, trauma, and/or pain is contacted directly with a composition described herein up to about one hour after trauma has occurred. In some embodiments, the site of bruising, trauma, and/or pain is contacted directly with a composition described herein up to about 24 hours, 48 hours, and/or 72 hours after trauma has occurred. In some embodiments, a composition described herein is orally administered to a subject post-operatively. In some embodiments, a composition described herein is administered to a subject about 24 hours, 48 hours, and/or 72 hours after surgery.

In another aspect, a combination of administration of a composition described herein both pre-operatively and post-operatively may provide additional therapeutic benefits to a subject in need thereof. In some embodiments, the combination comprises oral administration of a composition described herein. In some embodiments, the combination comprises directly contacting the site of bruising, trauma, and/or pain with a composition described herein. In some embodiments, the combination comprises both oral administration of a composition described herein and directly contacting the site of bruising, trauma, and/or pain with a composition described herein. In some embodiments, the site of bruising, trauma, and/or pain is contacted directly with a first composition pre-operatively and the site of bruising, trauma, and/or pain is contacted directly with a second composition post-operatively. In some embodiments a first composition is orally administered to the subject pre-operatively and the site of bruising, trauma, and/or pain is contacted directly with a second composition post-operatively. In some embodiments, the site of bruising, trauma, and/or pain is contacted directly with a first composition pre-operatively and a second composition is orally administered to the subject post-operatively. In some embodiments, a first composition is orally administered to the subject pre-operatively and a second composition is orally administered to the subject post-operatively. In some embodiments, the first composition and the second composition are different. In some embodiments, the first composition and the second composition are the same. For example, without wishing to be limited by any particular theory, the first composition comprises a total bromelain concentration of about 1% (w/w) and is applied directly to the site of bruising and/or trauma pre-operatively, and the second composition comprises a total arnica concentration of about 10% (w/w) and is applied directly to the site of bruising and/or trauma post-operatively.

Pharmaceutically Acceptable Compositions and Kits Comprising Arnica and/or Bromelain

In some aspects, the compositions comprising, consisting essentially of, or consisting of one or more agents selected from arnica and bromelain are formulated as pharmaceutically acceptable compositions. The pharmaceutical composition may be formulated with an effective amount of a composition as described herein. In certain embodiments, a pharmaceutical composition comprises a composition described herein. In certain embodiments, the pharmaceutical composition further comprises one or more topically acceptable adjuvants or excipients. In certain embodiments, the pharmaceutical composition further comprises one or more agents that increase transdermal absorption of the composition. In certain embodiments, the pharmaceutical composition further comprises dimethyl sulfoxide (DMSO).

In certain embodiments, a composition described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a painful condition in a subject in need thereof. In certain embodiments, the effective amount is an amount effective for preventing a painful condition in a subject in need thereof. In certain embodiments, the effective amount is an amount effective for treating bruising in a subject in need thereof. In certain embodiments, the effective amount is an amount effective for preventing or reducing the risk of bruise development as a result of surgery or trauma in a subject in need thereof. In certain embodiments, the effective amount is an amount effective for reducing the size, color, or swelling of a bruise that results from surgery in a subject in need thereof. In certain embodiments, the effective amount is an amount effective for reducing the size, color, or swelling of a bruise that results from trauma in a subject in need thereof. In certain embodiments, the effective amount is an amount effective for treating symptoms associated with surgery or trauma in a subject in need thereof.

In certain embodiments, the effective amount is an amount capable of inhibiting and/or reducing a proinflammatory response. In certain embodiments, the effective amount is an amount that reduces a proinflammatory response by at least about 5%, at least about 10%, at least about 20%, at least about 30%, at least about 40%, at least about 50%, at least about 60%, at least about 70%, at least about 80%, at least about 90%, at least about 95%, at least about 98%, or at least about 99% upon administration of a composition described herein. In certain embodiments, the effective amount is an amount capable of inhibiting and/or reducing the function or production of proinflammatory prostaglandins. For example, without wishing to be bound by any particular theory, bromelain has been shown to inhibit the prostaglandin synthetic pathway without the deleterious side effects associated with NSAIDS. Bromelain may act at the thromboxane synthetase step in the arachidonate cascade. In certain embodiments, the effective amount is an amount effective for promoting or inducing an anti-inflammatory response. In certain embodiments, an effective amount is an amount that increases an anti-inflammatory response by at least about 5%, at least about 10%, at least about 20%, at least about 30%, at least about 40%, at least about 50%, at least about 60%, at least about 70%, at least about 80%, at least about 90%, at least about 95%, at least about 98%, or at least about 99% upon administration of a composition described herein.

Pharmaceutical compositions described herein can be prepared by any method known in the art of pharmacology. In general, such preparatory methods include bringing a composition described herein (i.e., the “active ingredient” or “active ingredients”) into association with a carrier or excipient, and/or one or more other accessory ingredients, and then, if necessary and/or desirable, shaping, and/or packaging the product into a desired single- or multi-dose unit. In certain embodiments, a composition described herein further comprises a pharmaceutically acceptable adjuvant, excipient, or emollient.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) total active ingredients.

An “excipient” is a compound or substance formulated to confer therapeutic enhancement to the “active ingredient” (e.g., the composition comprising one or more active ingredients) in the pharmaceutical composition, such as, for example, facilitating drug absorption, reducing viscosity, or enhancing solubility. One of ordinary skill in the art will recognize that choosing an appropriate excipient depends on multiple factors, depending on, for example, route of administration, dosage formulation, the identity and properties of the active ingredient(s), and the like. Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition. The exact amount of a compound required to achieve an effective amount will vary from subject to subject, depending on, for example, species, age, and general condition of a subject, severity of the side effects or disorder, identity of the particular active ingredient(s), mode of administration, and the like. Remington's The Science and Practice of Pharmacy, 21^{st} Ed., A. R. Gennaro (Lippincott, Williams, & Wilkins, Baltimore, MD, 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the composition, its use is contemplated to be within the scope of this invention.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary preservatives include antioxidants, chelating agents, antimicrobial preservatives, antifungal preservatives, antiprotozoan preservatives, alcohol preservatives, acidic preservatives, and other preservatives.

An effective amount may be included in a single dose (e.g., single topical dose, single oral dose) or multiple doses (e.g., multiple topical doses, multiple oral doses, or a combination of topical and oral doses). In certain embodiments, when multiple doses are administered to a subject or applied to a tissue or to the site of bruising and/or trauma, any two doses of the multiple doses include different or substantially the same amounts of a composition described herein. In certain embodiments, when multiple doses are administered to a subject or applied to a tissue or to the site of bruising and/or trauma, the frequency of administering the multiple doses to the subject or applying the multiple doses to the tissue is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or to the site of bruising and/or trauma is one dose per day. In certain embodiments, the frequency of administering the multiple doses to the subject or applying the multiple doses to the tissue or to the site of bruising and/or trauma is two doses per day. In certain embodiments, the frequency of administering the multiple doses to the subject or applying the multiple doses to the tissue or to the site of bruising and/or trauma is three doses per day. In certain embodiments, when multiple doses are administered to a subject or applied to a tissue or to the site of bruising and/or trauma, the duration between the first dose and last dose of the multiple doses is one day, two days, three days, four days, five days, six days, one week, two weeks, three weeks, one month, two months, three months, four months, six months, nine months, one year, or the lifetime of the subject or tissue. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a composition described herein. In certain embodiments, a dose (e.g., a single dose, or any dose of multiple doses) described herein includes independently between 0.1 μL and 1 μL, between 0.001 mL and 0.01 mL, between 0.01 mL and 0.1 mL, between 0.1 mL and 1 mL, between 1 mL and 3 mL, between 3 mL and 10 mL, between 10 mL and 30 mL, between 30 mL and 100 mL, between 100 mL and 300 mL, between 300 mL and 1,000 mL, or between 1 L and 10 L, inclusive, of a composition described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is about 70 kg.

Compositions provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the condition being treated and the severity of the bruise and/or trauma; the activity of the specific active ingredient or ingredients employed; the specific composition employed; the age, body weight, general health, sex, and diet of the subject; the time of administration, route of administration, and rate of excretion of the specific active ingredient or ingredients employed; the duration of the treatment; drugs used in combination or coincidental with the specific active ingredient or ingredients employed; and like factors well known in the medical arts.

The compositions provided herein can be administered by any route, including enteral (e.g., oral), parenteral, subcutaneous, transdermal, intradermal, topical (as by powders, ointments, and/or creams), mucosal, nasal, bucal, sublingual; and/or as an aerosol. Specifically contemplated routes are topical administration and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In some embodiments, the composition described herein is suitable for topical administration to the a subject. In some embodiments, the compositions are formulated for transdermal administration. In some embodiments, the compositions are formulated for oral delivery. In some embodiments, the compositions are formulated as an aerosol. In some embodiments, the aerosol is formulated for application to the skin and/or spayed directly on the skin. In addition, two or more formulation types may be employed for the administration of the compositions described herein. For example, a composition may be formulated for oral delivery and administered to a patient pre-operatively, followed by contacting the site of bruising, trauma, and/or pain with a composition formulated for topical and/or transdermal administration post-operatively. Other combinations of oral and topical formulations for administration to a subject in need thereof are contemplated to be within the scope of this invention.

Dosage forms for topical and/or transdermal administration of a compound described herein may include emollients, ointments, pastes, creams, lotions, gels, powders, solutions, sprays, inhalants, and/or patches. In certain embodiments, a composition described herein is formulated for topical administration. In certain embodiments, a composition is cream, gel, ointment, or lotion. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of one or more active ingredients to the body. Such dosage forms can be prepared, for example, by dissolving and/or dispensing the active ingredient(s) in the proper medium. Alternatively or additionally, the rate can be controlled by either providing a rate controlling membrane and/or by dispersing the active ingredient(s) in a polymer matrix and/or gel.

An “emollient” is any compound, mixture, or agent designed to make the epidermis softer or more pliable. The term “emollient” also encompasses moisturizers. In general, an emollient increases the skins hydration by reducing evaporation. An emollient can be a mixture of components with different effects. For example, an emollient may contain an occlusive to help the skin retain moisture after the emollient is applied or a humectant to draw moisture from the air into the outer epidermal layers of the skin.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Liquid dosage forms for oral and parenteral administration include pharmaceutically acceptable emulsions, micro emulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof. Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The composition (i.e., the active ingredient(s)) can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

A composition as described herein can be administered in combination with one or more additional pharmaceutical agents (e.g., therapeutically and/or prophylactically active agents). The composition can be administered in combination with additional pharmaceutical agents that improves the composition's activity (e.g., potency and/or efficacy in treating bruising, pain, and/or trauma in a subject in need thereof, in preventing bruising and/or pain in a subject in need thereof, and/or in reducing the risk to develop a bruise in a subject in need thereof), improve bioavailability, improve safety, reduce drug resistance, reduce and/or modify metabolism, inhibit excretion, and/or modify distribution in a subject or tissue. In certain embodiments, a composition described herein is formulated to comprise one or more additional pharmaceutical agents (i.e., the additional pharmaceutical agent is not formulates or administered separately from the pharmaceutical composition). In certain embodiments, the additional agent(s) is one or more anesthetic agents selected from prilocaine, lidocaine, benzocaine, tetracaine, and proxymetacaine. In certain embodiments, the anesthetic agent is prilocaine. In certain embodiments, the anesthetic agent is lidocaine. In certain embodiments, the anesthetic agents are lidocaine and prilocaine. It will also be appreciated that the therapy employed may achieve a desired effect for the same disorder, and/or it may achieve different effects. In certain embodiments, a pharmaceutical composition described herein including a composition described herein and an additional pharmaceutical agent shows a synergistic effect that is absent in a pharmaceutical composition including one of the composition and the additional pharmaceutical agent, but not both.

Also encompassed by the disclosure are kits (e.g., pharmaceutical packs). The kits provided may comprise a composition described herein and a container (e.g., a vial, ampule, bottle, syringe, tube, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form. In certain embodiments, the kit comprises a single container comprising a composition described herein, wherein the composition is formulated for topical administration. In certain embodiments, the kit comprises a cream, gel, ointment, or lotion. In certain embodiments, the kit comprises an aerosol. In some embodiments, the kit comprises a composition formulated for oral administration. In some embodiments, the first container comprises a composition formulated for topical administration and the second container comprises a composition formulated for oral administration. In some embodiments, the two containers in the kit may be administered simultaneously. In some embodiments, the two containers in the kit may be administered at different times. For example, without wishing to be bound by an particular theory, an oral composition may be administered pre-operatively, followed by post-operative administration of a topical composition directly to the site of bruising, trauma, and/or pain.

Thus, in one aspect, provided are kits including a first container comprising a composition described herein. In certain embodiments, the kits are useful for treating bruising, pain, and/or trauma in a subject in need thereof. In certain embodiments, the kits are useful for preventing bruising and/or pain in a subject in need thereof. In certain embodiments, the kits are useful for reducing the risk of bruising and/or pain in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating bruising, pain, and/or trauma in a subject in need thereof. In certain embodiments, the kits and instructions provide for preventing bruising and/or pain in a subject in need thereof. In certain embodiments, the kits and instructions provide for reducing the risk of developing a bruising and/or pain in a subject in need thereof.

Thus, in one aspect the present disclosure provides a kit comprising a composition described herein; and instructions for administering to a subject or contacting a biological sample with the composition. In certain embodiments, the kit comprises a composition comprising, consisting essentially of, or consisting of one or more agents selected from arnica and bromelain, and optionally an agent that increases transdermal absorption and/or one or more anesthetic agents selected from prilocaine, lidocaine, benzocaine, tetracaine, and proxymetacaine; and instructions for administering to a subject or contacting a biological sample with the composition. In certain embodiments, the kit further comprises a topically acceptable adjuvant or emollient

### EXAMPLES

In order that the invention described herein may be more fully understood, the following examples are set forth. The examples described in this application are offered to illustrate the methods, compositions, and systems provided herein and are not to be construed in any way as limiting their scope.

### Example 1—Bruise Induction in an Ex Vivo Tissue Sample

Tissue samples obtained during an abdominoplasty or other surgical procedure are maintained ex vivo. One tissue sample provides one dose group to be tested with a composition described herein. Each dose group is comprised of 7 separate assays using a composition described herein, increasing the statistical significance of measurements. A bruise can be induced by the injection of 0.25-25 μL of the patients' blood intradermally into the abdominoplasty explanted tissue. The tissue sample is then maintained at 370 in warm phosphate buffered saline (PBS) solution. The tissue specimen (i.e., the dose group) is monitored and assessed over a 72 hour period. Assessments of bruise size, color, and severity are made at various intervals, for example, immediately following the injection, and at 12 hour, 24 hour, 36 hour and 48 hour intervals post-injection. Topical formulations of compositions provided herein are applied within 30 minutes from intradermal injection. In addition, the effect of these compositions on preventing or reducing the severity of bruising, trauma, and/or pain can be assessed using pre-treatment groups, which have the topical formulation applied to the assay area 1 hour before bruise induction (e.g., intradermal injection).

### Example 2—Assessing Bruise Size, Color, and Severity

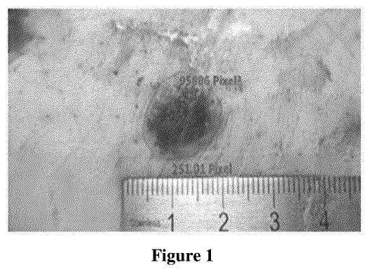

The color of a bruise in an ex vivo tissue sample or in vivo in a subject is graded on the following scale to indicate severity, where 1 is the least severe and 4 is the most severe: 1) no discoloration beyond that of injected blood, 2) mild discoloration, 3) moderate discoloration, and 4) severe discoloration. Severe discoloration may serve as a control in the sample (e.g., tissue sample or subject), such that a decrease in severe discoloration indicates efficacy of the composition. FIG. 1 shows the control discoloration in an abdominoplasty specimen after injection of 25 μL of patient blood intradermally. Densitometry or a visual scale of discoloration can be used to categorize bruise color of each sample.

Bruise size can be monitored and measured using photomicrography using microscopy and AxioVision Microscopy (Carl Zeiss, Inc.) software to measure bruise area in pixels, which is then converted into cm².

The swelling associated with bruise area (i.e., bruise size) can be categorized according to severity as follows, with 1 being the least severe and 3 being the most severe: 1) no swelling, 2) mile swelling, and 3) moderate swelling. Measurements for swelling are made digitally by palpitating the bruise area at various time points, and compared to a control group. A composition that decreases swelling compared to the control, which shows a moderate swelling level upon intradermal injection of blood in a tissue sample, will be considered significant when p<0.05, with swelling data expressed as mean±standard deviation (SD). The swelling data obtained is compared using one way (time) variance analysis, followed by a Fisher test, if necessary.

The measurement criteria for bruise size, color, or severity in a tissue sample can also be applied directly to a subject. A clinically significant decrease in bruise size, color, or swelling severity indicates that a composition is effective at treating bruising and/or trauma.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims. Where the claims or description relate to a product (e.g., a composition of matter), it should be understood that methods of making or using the product according to any of the methods disclosed herein, and methods of using the product for any one or more of the purposes disclosed herein, are encompassed by the present disclosure, where applicable, unless otherwise indicated or unless it would be evident to one of ordinary skill in the art that a contradiction or inconsistency would arise. Where the claims or description relate to a method, it should be understood that product(s), e.g., compositions of matter, device(s), or system(s), useful for performing one or more steps of the method are encompassed by the present disclosure, where applicable, unless otherwise indicated or unless it would be evident to one of ordinary skill in the art that a contradiction or inconsistency would arise.

Where ranges are given herein, embodiments are provided in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, embodiments that relate analogously to any intervening value or range defined by any two values in the series are provided, and that the lowest value may be taken as a minimum and the greatest value may be taken as a maximum. Where a phrase such as “at least”, “up to”, “no more than”, or similar phrases, precedes a series of numbers herein, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated where applicable. A reasonable lower or upper limit may be selected or determined by one of ordinary skill in the art based, e.g., on factors such as convenience, cost, time, effort, availability (e.g., of samples, agents, or reagents), statistical considerations, etc. In some embodiments an upper or lower limit differs by a factor of 2, 3, 5, or 10, from a particular value. Numerical values, as used herein, include values expressed as percentages. For each embodiment in which a numerical value is prefaced by “about” or “approximately”, embodiments in which the exact value is recited are provided. For each embodiment in which a numerical value is not prefaced by “about” or “approximately”, embodiments in which the value is prefaced by “about” or “approximately” are provided. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. In some embodiments a method may be performed by an individual or entity. In some embodiments steps of a method may be performed by two or more individuals or entities such that a method is collectively performed. In some embodiments a method may be performed at least in part by requesting or authorizing another individual or entity to perform one, more than one, or all steps of a method. In some embodiments a method comprises requesting two or more entities or individuals to each perform at least one step of a method. In some embodiments performance of two or more steps is coordinated so that a method is collectively performed. Individuals or entities performing different step(s) may or may not interact.

Section headings used herein are not to be construed as limiting in any way. It is expressly contemplated that subject matter presented under any section heading may be applicable to any aspect or embodiment described herein.

Embodiments or aspects herein may be directed to any agent, composition, article, kit, and/or method described herein. It is contemplated that any one or more embodiments or aspects can be freely combined with any one or more other embodiments or aspects whenever appropriate. For example, any combination of two or more agents, compositions, articles, kits, and/or methods that are not mutually inconsistent, is provided. It will be understood that any description or exemplification of a term anywhere herein may be applied wherever such term appears herein (e.g., in any aspect or embodiment in which such term is relevant) unless indicated or clearly evident otherwise.

1. A method for treating bruising and/or trauma, the method comprising contacting the site of bruising and/or trauma of a tissue sample with an effective amount of a composition comprising one or more agents selected from arnica bromelain, quercetin, and apigenin.

2. The method of claim 1, wherein the composition comprises two or more agents selected from arnica, bromelain, quercetin, and apigenin.

3. The method of claim 1, wherein the composition comprises three or more agents selected from arnica, bromelain, quercetin, and apigenin.

4. The method of claim 1, wherein the composition comprises four agents selected from arnica, bromelain, quercetin, and apigenin.

5. The method of claim 1, wherein the tissue is in vivo or ex vivo.

6. A method for treating bruising and/or trauma, the method comprising administering to a subject in need thereof a composition comprising one or more agents selected from arnica, bromelain, quercetin, and apigenin, wherein the site of bruising and/or trauma is contacted directly with the composition and/or the composition is orally administered to the subject.

7. The method of claim 6, wherein the composition comprises two or more agents selected from arnica, bromelain, quercetin, and apigenin.

8. The method of claim 6, wherein the composition comprises three or more agents selected from arnica, bromelain, quercetin, and apigenin.

9. The method of claim 6, wherein the composition comprises all four agents selected from arnica, bromelain, quercetin, and apigenin.

10.-11. (canceled)

12. A method for treating pain, the method comprising administering to a subject in need thereof a composition comprising one or more agents selected from arnica and bromelain, wherein the site of pain is directly contacted with the composition and/or the composition is orally administered to the subject.

13. The method of claim 12, wherein the composition comprises two or more agents selected from arnica, bromelain, quercetin, and apigenin.

14. The method of claim 12, wherein the composition comprises three or more agents selected from arnica, bromelain, quercetin, and apigenin.

15. The method of claim 12, wherein the composition comprises four agents selected from arnica, bromelain, quercetin, and apigenin.

16. A composition comprising one or more agents selected from arnica, bromelain, quercetin, and apigenin, and one or more anesthetic agents selected from prilocaine, lidocaine, benzocaine, tetracaine, and proxymetacaine, and optionally a topically acceptable adjuvant or emollient.

17. The composition of claim 16 comprising two or more agents selected from arnica, bromelain, quercetin, and apigenin, and one or more anesthetic agents selected from prilocaine, lidocaine, benzocaine, tetracaine, and proxymetacaine, and optionally a topically acceptable adjuvant or emollient.

18. The composition of claim 16 comprising three or more agents selected from arnica, bromelain, quercetin, and apigenin, and one or more anesthetic agents selected from prilocaine, lidocaine, benzocaine, tetracaine, and proxymetacaine, and optionally a topically acceptable adjuvant or emollient.

19. The composition of claim 16 comprising all four agents selected from arnica, bromelain, quercetin, and apigenin, and one or more anesthetic agents selected from prilocaine, lidocaine, benzocaine, tetracaine, and proxymetacaine, and optionally a topically acceptable adjuvant or emollient.

20. (canceled)
